(19) United States
(12) Patent Application Publication
(10) Pub. No.: US 2010/0047428 A1
(43) Pub. Date: Feb. 25, 2010

(54) METHOD FOR THE EXTRACTION OF ONE OR SEVERAL PROTEINS IN MILK

(75) Inventors: Alain Lejars, Pierres (FR); Michel Nogre, Vanves (FR); Michael Tellier, Eaubonne (FR)

Correspondence Address: MARSH, FISCHMANN & BREYFOGLE LLP, 8055 East Tufts Avenue, Suite 450, Denver, CO, 80237, US

(73) Assignee: LFB BIOTECHNOLOGIES, Les Ulis (FR)

(21) Appl. No.: 12/300,495

(22) PCT Filed: May 31, 2007
(86) PCT No.: PCT/FR07/00908

§ 371 Date: May 27, 2009

(30) Foreign Application Priority Data
May 31, 2006 (FR) 0604864

(51) Int. Cl. A23C 7/04; C07K 14/745; A23C 9/20

(52) U.S. Cl. 426/580; 530/381; 530/384

(57) ABSTRACT

The invention is related to a process for extracting at least one protein present in milk, said protein exhibiting an affinity for the complexed or non-complexed calcium ions of said milk, comprising the following steps consisting of:
a) releasing the protein by precipitation of calcium compounds obtained by contacting the milk with a soluble salt, the anion of which is selected for its capability to form said insoluble calcium compounds in such a medium, in order to obtain in this way a protein-enriched liquid phase,
b) separating the protein-enriched liquid phase from the precipitate of calcium compounds, said liquid phase being, moreover, separated in a lipidic phase and in a non-lipidic aqueous phase comprising the protein, and
c) recovering the non-lipidic aqueous phase comprising the protein.

The present invention is related to a process of extraction of one or more proteins present in the milk, said proteins exhibiting an affinity for complexed or non-complexed calcium ions of said milk.

In the context of the invention, complexed or non-complexed calcium ions refer to either phosphocalcic salts bound to caseins in order to form a micellar colloidal structure of caseins, or to such salts not bound to caseins and which are, therefore, free. Such ions are also the different calcium salts and/or organic and/or inorganic calcium complexes different of those mentioned here-above, soluble in the milk. Proteins exhibiting an affinity for calcium ions of the milk represent those which are there naturally present, such as lactalbumins, lactoglobulins and immuno-globulins. These proteins can also represent recombinant proteins present in the milk of transgenic animals, for example, blood clotting factors, in particular Factor VII, Factor VIII and Factor IX.

The major part of commercially available medicaments corresponds to chemical substances obtained by synthesis. As a matter of fact, until recently, the modern medicine relied highly on medicaments produced by chemical synthesis for treating or for diagnosis of diseases.

However, the proteins represent a substantial part of the molecules carrying a biological information. This is especially the case of a large number of hormones, growth factors, blood clotting factors or antibodies.

Generally, proteins are polymers on aminoacid basis, mostly of high molecular weight, which cannot be obtained by chemical synthesis with acceptable costs. Such proteins for therapeutic use are usually isolated and purified from, for example, living organisms, human or animal tissues or blood. This is the case especially of insulin extracted from pig pancreas, of blood clotting factors, such as Factor VIII or Factor IX, extracted from blood plasma, or immunoglobulins.

Although the processes of preparation of the here-above proteins are largely used at present, they have, however, drawbacks. The low content of some proteins, such as erythropoietin, extracted from blood platelets, does not allow their isolation in sufficient amounts to meet the constantly increasing therapeutic needs. In addition, the presence of viruses, prions or other pathogenic agents in the human plasma requires the inclusion of additional virus inactivation and/or virus elimination steps into the manufacturing processes of plasma proteins, in order to obtain products useful in therapeutics.

In order to overcome these drawbacks, use is made of genetic engineering, a technique largely used also in the synthesis of a protein from an isolated gene, transferred into a cell, which is in charge of the secretion of the considered protein. Such a protein, obtained outside of its original cellular system, is called <<recombinant >>.

According to this technique, different cellular systems can be used.

Bacterial systems, for example E. coli, are largely used and efficient. They allow to produce recombinant proteins at low cost. However, such systems are limited to the preparation of simple, non glycosylated proteins, which do not require elaborate folding processes.

The fungal systems are equally used for the production of secreted proteins. The drawback of these fungal systems lies in the fact that they are at the origin of the post-translational modifications, consisting of, for example, grafting glycan moieties and sulfate groups, which highly affect the pharmacokinetic properties of the produced proteins, especially by addition of various groups of mannose derivatives.

The systems using baculovirus allow to produce very different proteins, such as vaccinal proteins or the growth hormone, but their application on industrial scale is not optimized.

Use is also made of mammal cells culture for the preparation of recombinant complex proteins, such as monoclonal antibodies. The cell expression systems lead to correctly folded and modified recombinant proteins. The low yield comparing to the manufacturing costs is a major drawback.

A variant of such cell systems consists of carrying out transgenic plants for obtaining proteins in great amounts. These systems however generate plant-specific post-translational modifications, in particular by addition of highly immunogenic xylose residues to the produced proteins, thus limiting their use in therapeutic applications.

An alternative of the previously mentioned cell systems consists of the use of transgenic animals for producing recombinant vaccines or complex therapeutic proteins. The thus obtained proteins exhibit a glycosylation close to that of man and are correctly folded. These complex proteins are not only consisting of only one simple polypeptide chain, such as, for example, the growth hormone, but they are modified in different ways after assembling the aminoacids, especially by specific cleavages, glycosylations and carboxymethylations. In the large majority of cases, the modifications cannot be carried out by bacterial cells or yeasts. On the other hand, the transgenic animals allow to combine both the expression levels encountered in bacterial cell systems and the post-translational modifications obtained by means of cell cultures, while reducing the manufacturing costs compared with the use of cell expression systems.

Among the biological materials from transgenic animals, the milk is the subject of studies leading to consider it as a source of a very satisfactory secretion of recombinant proteins.

The recombinant proteins, produced from the milk of transgenic animals, can be easily obtained by grafting the gene encoding the protein of interest onto the regulatory region of one of the genes responsible for the synthesis of milk proteins, which will direct the synthesis specifically in the mammary gland, then its secretion into the milk.

By way of example, the application EP 0 527 063 describing the production of a protein of interest in the milk of a transgenic mammal, the expression of the gene encoding the protein of interest being controlled by a promoter of a protein of lactoserum, can be mentioned.

Further patent applications or patents describe the preparation of antibodies (EP 0 741 515), of collagen (WO 96/03051), of human Factor IX (U.S. Pat. No. 6,046,380 and of Factor VIII/von Willebrand Factor complexes (EP 0 807 170), in the milk of transgenic mammals.

In spite of the satisfactory results of these methods in terms of protein expression, the use of milk as source of recombinant proteins has drawbacks. The major drawback lies in the difficulties, on one hand, of their extraction from the milk with a satisfactory yield and, on the other hand, in their subsequent purification.

Indeed, the milk is a mixture consisting to 90% of water comprising various constituents which can be grouped in three categories. The first category, called lactoserum (or whey), consists of glucides, soluble proteins, minerals and water-soluble vitamins. The second category, called lipidic phase (or cream), contains fats in form of emulsion. The third category, called proteic phase, consists of about 80% of caseins, which form a whole of precipitable proteins at a pH of 4.6 or under the action of the rennet, enzymatic coagulant, in the presence of calcium. The different caseins form a colloidal micellary complex, able to reach diameters of about 0.5 μm, with phosphocalcic salts, appearing for example in form of aggregates (<<clusters>>) of tricalcium phosphate, that is Ca₉(PO₄)₆. Such micelles are formed from casein sub-units consisting of casein-K-rich hydrophilic layer surrounding a hydrophobic nucleus, the phosphocalcic salts being bound by electrostatic interactions onto the hydrophilic layer. These phosphocalcic salts can also be present in the internal volume of the micelle without being bound to the casein. This proteic phase contains also soluble proteins, such as lactalbumins and lactoglobulins, and albumins and immunoglobulins resulting from blood.

Depending upon the nature of the recombinant protein secreted into the milk of transgenic animals, it can be present in the lactoserum or in the proteic phase, even in both at the same time. The abundance and the complexity of each category of milk constituents makes all the more difficult to carry out an extraction of this protein, especially when it is trapped in the casein micelles. A further difficulty lies in the fact that the presence of this protein in one of both phases is not foreseeable with certainty.

A recombinant protein can also exhibit affinities for the calcium ions of the milk which are present in form either of salts and/or various soluble complexes, or of phosphocalcic salts of the casein micelles. These affinities are expressed by electrostatic linkages between the protein and the bivalent calcium cations. The affinities protein/calcium ions allow to define the affinity constants which, depending upon their value, determine the binding strength. Generally speaking, the major part of proteins exhibiting an affinity for the calcium ions is bound to the phosphocalcic salts of the micelles. The extraction thereof requires to carry out complex steps, bound with problems of implementation and of yield.

The classical solution used in the diary industry for isolating proteins consisting of a pasteurisation, followed by an enzymatic coagulation or acidic precipitation (pH 4.6), cannot be applied to this case, because the recombinant proteins are often denaturated under the combined effect of the temperature and the pH. In addition, the trapping of the proteins in the casein micelles leads to low extraction yields. Further solutions consisting in performing physical methods of fractionation of the milk by filtration, centrifugation and/or sedimentation or precipitation techniques, lead also to unacceptable extraction yields and to extracted recombinant proteins with a poor purity.

The document EP 0 264 166 describes the secretion of a desired protein into the milk of genetically transformed animals. Purification steps of this protein from milk are not mentioned in that document.

The U.S. Pat. No. 4,519,945 describes a process for extraction of a recombinant protein by preparing a precipitate of caseins and of lactoserum from milk, performing the steps of acidification and of heating as mentioned previously. This process generates a significant loss of activity of the considered protein and a low extraction yield.

The U.S. Pat. No. 6,984,772 discloses a process for purification of recombinant fibrinogen from the milk of a transgenic mammal. This process includes a step of separation of the lactoserum from the casein pellet and of the proteic phase by successive centrifugations. The lactoserum is isolated, then stored for the subsequent processing, resulting in a purified solution of fibrinogen.

However, this process cannot be applied to the production with a satisfying yield of recombinant proteins trapped in and/or on the casein micelles, such as plasma clotting factors, for example, Factor VII, Factor VIII and Factor IX.

The patent application WO 2004/076695 describes a filtration process of recombinant proteins from the milk of transgenic animals. This process includes a first step of clarification of the milk, that is a step consisting in eliminating milk components in a way to obtain a solution liable to be filtered through a filter membrane exhibiting a pore size of 0.2 μm diametre. Such a step ends in the elimination of casein micelles. Consequently, the carrying out of this step can be redhibitory, in terms of yield, if the casein micelles are liable to contain a protein of interest trapped within their structure.

The U.S. Pat. No. 6,183,803 describes a process for isolating proteins naturally present in the milk, such as lactalbumins, and recombinant proteins, for example human albumin or α1-antitrypsin, from milk. This process includes an initial step of contacting the milk comprising a protein of interest with a chelating agent. This generates the destructuration of the casein micelles, leading to a clarified milk serum comprising the caseins, the proteins of the lactoserum and the protein of interest. The process further comprises a step of restructuration of the casein micelles by addition, to the liquid medium (clarified milk serum), of insoluble salts of divalent cations. These micelles precipitate, what results in a liquid phase comprising the protein of interest, which is not trapped in the micelles, because the salts saturate the electrostatic linkage sites of caseins. According to this process, the separation of the protein of interest is, therefore, finally carried out by restructuration of micelles and their precipitation.

This process is complex to carry out and cannot be applied to proteins having a relatively high affinity for the calcium ions. The proteins of the coagulation, and especially those known as being synthetized under the influence of the vitamine K, enter into this category.

Starting from a double observation that the separation and purification processes of certain categories of recombinant proteins secreted into the milk of transgenic animals present in the lactoserum lead to very low yields, and of those of other protein categories which are trapped in the casein micelles, are complex to be carried out, the Applicant set himself the task to provide for a process of extraction, from milk, of milk constitutive proteins, natural or non natural, such as the recombinant Factor VII, Factor VIII and Factor IX, exhibiting an affinity for the ionic forms of calcium of the milk, with a simplified implementation, leading moreover to a satisfactory production yield, while retaining the biological activity of the protein.

Thus, the invention concerns a process for extracting at least one protein present in milk, said protein exhibiting an affinity for the complexed or non-complexed calcium ions of said milk, including the following steps consisting in:
a) releasing the protein by precipitation of calcium compounds obtained by contacting the milk with a soluble salt, the anion of which is selected for its ability to form, in such medium, the said insoluble calcium compounds, in order to obtain in this way a protein-enriched liquid phase,
b) separating the protein-enriched liquid phase of the precipitate of calcium compounds, said liquid phase being moreover separated in a lipidic phase and in an aqueous non-lipidic phase comprising the protein, and
c) recovering the aqueous non-lipidic phase comprising the protein.

The Applicant has surprisingly noted that the fact of adding a soluble salt, the anion of which is selected for its capability to form precipitates of calcium compounds in the milk containing a protein, especially a recombinant protein, exhibiting an affinity for the complexed or non-complexed calcium ions, that is exhibiting sites of fixation to the calcium ions, allows to precipitate the calcium compounds, whereas the protein of interest is released from these complexed or non-complexed ions and is found again in solution in the liquid phase.

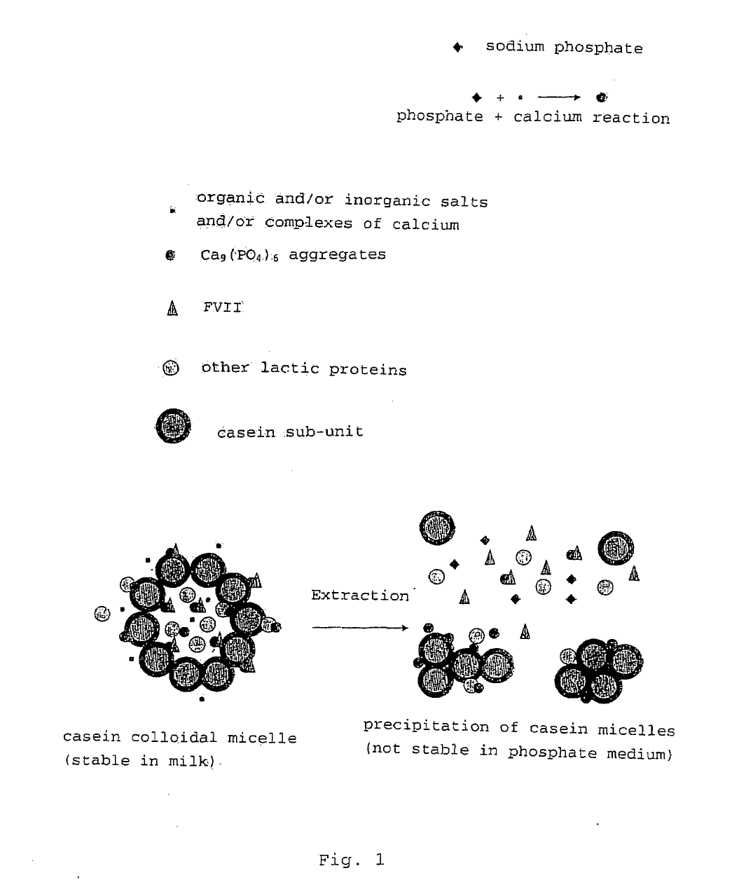

The complexed or non-complexed calcium ions, as mentioned here-above, represent the different organic and/or inorganic calcium salts and/or complexes, soluble in the milk. These salts or complexes can be present in the internal volume of the casein micelle (see hereafter on the FIG. 1).

These calcium ions represent also the phosphocalcic salts interacting with the casein micelles, especially in form of aggregates (<<clusters>>). These salts are also present in the milk in form of monocalcic phosphate and/or dicalcic phosphate, which are in equilibrium with the other ionic forms of calcium, depending upon the implemented chemical and biochemical reactions.

Finally, these calcium ions represent calcium/casein complexes, that is, represent sub-units of casein with which are associated, by electrostatic interaction, the phosphocalcic salts. These calcium/casein complexes refer also to the casein micelles associated with the phosphocalcic salts and with the organic and/or inorganic soluble calcium salts and/or complexes.

Insoluble calcium compounds refer to calcium salts or complexes, the solubility of which in the milk is less than 0.5%.

In the majority of cases, the proteins of interest will be associated mostly to phosphocalcic salts of the casein micelles.

Thus, protein exhibiting an affinity for complexed or non-complexed calcium ions refers to any protein having a sufficient number of fixation sites for calcium ions in order to be associated therewith, totally or partially, or to be associated, totally or partially, with the phophocalcic salts of the casein micelles.

By way of example, in the case of proteins of interest exhibiting numerous fixation sites for the calcium ions, for example 8 to 10 GLA domains, which are domains rich with γ-carboxyglutamic acids allowing to fix the calcium ions, from at least 70% to 90% of proteins of interest are trapped in and/or on the casein micelles. For other proteins exhibiting less fixation sites for the calcium ions, for example 2 to 8 GLA domains, at least 30% to 60% thereof are trapped in and/or on the casein micelles. Finally, even proteins exhibiting very few fixation sites for the calcium ions, for example 0 to 2 GLA domains, are likely to be trapped in and/or on the casein micelles, for example at a rate of at least 5% up to 20%. Such amounts of trapped proteins are not negligible for the implementation of a process on industrial scale, which implies the fact to reach the highest possible yield. The remaining proteins of interest, not trapped in and/on the micelles, exhibit an affinity for the other forms of calcium ions in the milk, mentioned here-above.

Consequently, the process of the invention can be applied to extraction of proteins from at least 2% to 10%, or from at least 40% to 60%, or, in particular, at least 90%, of which are associated to these calcium ions.

Such an affinity of the protein for the calcium ions can result from interactions of the not modified or in vivo or in vitro modified protein, for example, by post-translational modifications.

Thus, the proteins exhibiting numerous sites of fixation for the complexed or non-complexed calcium ions, can be associated with different forms of calcium present in the milk.

Not being bound to any interpretation of the observed mechanisms, the Applicant assumes that the addition of the soluble salt displaces the equilibrium of the phosphocalcic salts of the micelles, especially the calcium/phosphate ratio, causing thus their destructuration and the precipitation of aggregates of the casein sub-units. The proteins of interest associated with the phosphocalcic salts trapped in and/or on the micelles are released into the medium upon this destructuration. In addition, the proteins of interest are also released or dissociated from the phosphocalcic salts because these precipitate as insoluble calcium compounds under the effect of the soluble salt used in the process of the invention. Likewise, the proteins of interest which can also be associated with the soluble organic and/or inorganic calcium salts or complexes will also be dissociated, by the same type of reaction.

An example of such a mechanism is illustrated on the FIG. 1.

In the frame of the invention, the soluble salt represents any salt allowing to obtain the desired effect.

The soluble salt used in the process of the invention can be added to the milk in a concentration selected by those skilled in the art, in order to achieve the release of the protein from these interactions with the calcium ions. As such, it is a matter of a concentration sufficient to allow the release of at least 20% or, advantageously, from at least 30% to 50%, of the proteins of interest. In a particularly advantageous way, it is a matter of a concentration sufficient to allow the separation of at least 60% to 80%, or of at least 90%, of the proteins of interest.

In addition, the process of the invention can also be applied to proteins, among which a part only exhibits sites of fixation for calcium ions. For example, the process of the invention can be applied to the extraction of proteins contained in the milk, 1% of the whole of which is associated with calcium ions. The process can also be applied to proteins from at least 2% to 10%, or from at least 40% to 60% or, especially, at least 90% of which are associated with these calcium ions.

The process of the invention allows the precipitation especially of the aggregates of casein sub-units. This precipitation is due to the destructuration of the casein micelles, as mentioned above. The implementation of the process of the invention destabilizes, by precipitation, the colloidal state of the milk.

Consequently, the process of the invention is a process allowing the transition of the milk from a colloidal state to a liquid state, what corresponds to a direct extraction colloids/liquids.

The process of the invention allows also to obtain the lactoserum and the lipidic phase, with a lighter coloration than that of the starting milk. As a matter of fact, these are the calcium ions-bound caseins which confer their white color to the milk. Once precipitated, they are not able anymore to confer their color to the milk.

The process of the invention has therefore several advantages first, it can be very easily implemented, since it allows the separation of the proteins of interest by means of a simplified implementation. In addition, it allows to recover the proteins of interest in the non-lipidic aqueous phase with a very good yield. Advantageously, the process for extraction of the invention leads to yields of at least 50%, or of at least 60%, or yet, of at least 80%. In a particularly advantageous way, the yields are of at least 90%.

This process will also allow to obtain the non-lipidic aqueous phase comprising the protein of interest in a compatible form with the implementation of further purification steps thereof, especially by chromatography.

Finally, the proteins of interest are still biologically active, as the steps of the process of the invention are carried out at a pH not altering their biological activity. The pH is advantageously basic, for example about 8.

Soluble salt according to the invention refers to a salt with a solubility in the milk of at least 0.5 parts of salt per part of milk (w/w).

Advantageously, the soluble salt used in the process, is a phosphate salt. The salt can be in an aqueous solution which is added to the milk, or can be added directly to the milk in powder form.

Preferably, the phosphate salt is selected from the group consisting of sodium phosphate, lithium phosphate, potassium phosphate, rubidium phosphate and cesium phosphate, and is, in particular, sodium phosphate.

Alternatively, the soluble salt used for the implementation of the process of the invention can be an alcali metal oxalate, particularly sodium or potassium oxalate, or an alcali metal carbonate, in particular sodium or potassium carbonate, or a mixture thereof.

Advantageously, the concentration of the soluble salt in the aqueous solution, which is prepared for the implementation of the process, is comprised between 100 mM and 3 M, more preferably, between 200 mM and 500 mM and, in particular, between 200 mM and 300 mM.

Thus, according to a preferred embodiment of the invention, the soluble salt of the invention is the sodium phosphate, the concentration of which in aqueous solution is comprised between 100 mM and 3 M, more preferably between 200 mM and 500 mM and, in particular, between 200 mM and 300 mM.

The milk containing the protein of interest to be extracted can be raw non skimmed milk or skimmed milk. The advantage of applying the process of the invention to skimmed milk lies in the fact that the lipid content thereof is lower. The process can also be applied to fresh or frozen milk.

The step b) allows the separation of the liquid phase in a lipidic phase and a non-lipidic aqueous phase comprising the protein, what is preferably carried out by centrifugation. The non-lipidic aqueous phase is assimilated to lactoserum. This separation step allows also to isolate the aggregates of the micellary sub-units of caseins and the precipitate of calcium compounds.

The non-lipidic aqueous phase comprising the protein is separated from the lipidic phase.

This step allows to obtain advantageously a clear, non-lipidic aqueous phase.

Moreover, the process can include, following to the step c), a step of filtration of the non-lipidic aqueous phase carried out successively on filters with a decreasing porosity, preferably, of 1 μm, then of 0.45 μm. The use of these filters, such as on glass fibers basis, allows to reduce the content of the possibly still present lipids, fat globules and phospholipids naturally present in the milk. A porosity of less than 0.5 μm allows to maintain the bacteriological quality of the non-lipidic aqueous phase, and the later implemented purification supports (ultrafilters, chromatographic columns, etc.) (see hereafter). The lipidic phase is preferably filtered through these filters which retain completely the fat globules of the milk, and the filtrate is clear.

This step can be followed by a step of concentration/dialysis by ultrafiltration.

The concentration allows to reduce the volume of the non-lipidic aqueous phase in order to preserve it. The ultrafiltration membrane is selected by those skilled in the art depending upon the characteristics of the protein of interest. Generally speaking, a porosity limit with a pore size less or equal to the molecular weight of the protein of interest allows to concentrate the product without noticeable losses. For example, a membrane with a pore size of 50 kDa allows to concentrate FVII having a molecular weight of 50 kDa without losses.

The dialysis is intended to the conditioning of the aqueous phase of proteins for the possible further purification steps, especially by chromatography. It allows also to remove the small molecular weight components, such as lactoses, salts, peptides, proteoses peptones and any agent being able to harm the preservation of the product.

Preferably, the dialysis buffer is a 0.025 M-0.050 M sodium phosphate solution, pH 7.5-8.5.

The non-lipidic aqueous phase obtained after the step c) or, if the case arises, obtained following the steps of filtration and/or of concentration/dialysis, can be frozen and stored at a temperature of −30° C. until the implementation of the further purification steps thereof.

The process of the invention allows in this way the extraction and, if the case arises, the separation of one or more proteins of interest from calcium ions of the milk to which they are bound by electrostatic interactions.

The protein can be a protein naturally present in the milk, and represents, by way of example, β-lactoglobulin, lactoferrin, α-lactalbumin, immunoglobulins or proteoses peptones, or mixtures thereof.

The protein can also be a protein non naturally present in the milk. By way of example, Factor VII, Factor VIII, Factor IX, Factor X, alpha-1-anti-trypsin, anti-thrombin III, albumin, fibrinogen, insulin, myeline basic protein, proinsulin, plasminogen tissular activator et antibodies can be listed.

Thus, according to a preferred embodiment of the invention, the milk containing the protein of interest is a transgenic milk.

As a matter of fact, the proteins non naturally present in the milk could be synthetized therein by non-human transgenic mammals, thanks to the recombinant DNA techniques and to the transgenesis.

These techniques, well known to those skilled in the art, allow to synthetize any protein of interest in the milk of a transgenic animal.

Such a protein is then a recombinant or transgenic protein, these two terms being considered to be equivalent in the present application, synthetized by the recombinant DNA techniques.

<<Transgenic animal>> refers to any non-human animal having incorporated in the genome thereof an exogene DNA fragment, especially encoding a protein of interest, this animal expressing the exogenous DNA encoded protein and liable to transmit the exogenous DNA to its progeny.

As such, any non-human mammal is adapted to the production of such a milk.

Advantageously, use can be made of female rabbit, sheep, goat, cow, pig and mouse, this list is not limitative.

The secretion by the mammary glands of the protein of interest, allowing the secretion into the milk of the transgenic mammal, implies the control of the expression of the recombinant protein in a tissue-dependant fashion.

Such methods of control are well known to those skilled in the art. The control of the expression is performed thanks to sequences permitting the expression of the protein towards a particular tissue of the animal. These sequences are especially promoter sequences, and peptide signal sequences, as well.

Examples of promoters known to those skilled in the art are the WAP promoter (whey acidic protein), the casein promoter, the β-lactoglobulin promoter, this list is not limitative.

A manufacturing process of a recombinant protein in the milk of a transgenic animal can include the following steps: a synthetic DNA molecule comprising a gene encoding a protein of interest, this gene being under the control of a promoter of a protein naturally secreted into the milk, is integrated into the embryo of a non-human mammal. Afterwards, the embryo is placed into a mammal female of the same species, which gives birth to a transgenic animal. Once this subject is sufficiently developed, the lactation of the mammal is induced, next, the milk is collected. Then, the milk contains the recombinant protein of interest.

An example of process for preparing proteins in the milk of a mammal female other than a human being is described in the document EP 0 527 063, the teaching of which can be referred to for the production of the protein of interest of the invention.

A plasmid containing the WAP promoter is prepared by introduction of a sequence comprising the promoter of the WAP gene, this plasmid is prepared in a way to be able to receive a foreign gene placed under the dependence of the WAP promoter. The gene encoding a protein of interest is integrated and placed under the dependence of the WAP promoter. The plasmid containing the promoter and the gene encoding the protein of interest are used for obtaining transgenic animals, for example female rabbits, by microinjection into the male pronucleus of rabbit embryos. Afterwards, the embryos are transferred into the oviduct of hormonally prepared females. The presence of the transgenes is revealed by the technique of Southern from DNA extracted from the obtained transgenic young rabbits. The concentrations in the milk of animals are evaluated by specific radioimmunologic assays.

Advantageously, the protein produced in the milk and extracted according to the process of the invention is a clotting protein, or clotting factor. Indeed, it is known that such proteins exhibit a strong affinity for the calcium ions (Hibbard et al. (1980), J. Biol. Chem. 1980, Jan. 25; 255(2):638-645). According to a particular aspect of the invention, the clotting factor is activated during the process of extraction of the invention. It can be a matter especially of proteins <<vitamin K dependent>>, which are factors essential to the blood coagulation.

Advantageously, the protein produced in the milk and extracted according to the process of the invention is a protein containing the <<GLA-domains>>, which are able to fix the calcium ions, or the proteins containing the <<EGF domain>> (epidermal growth factor) or further domains identified as having the capability to fix the calcium ions, such as structures said in <<main EF>> (helix-loop-helix allowing to fix the calcium ion).

Moreover, the calcium dependent proteins are also proteins liable to be purified by the process of the invention, especially antibodies or monoclonal antibodies.

Advantageously, the protein of the invention is selected from the group consisting of Factor II (FII), Factor VII (FVII), Factor IX (FIX) and Factor X (FX), and their activated forms as well, protein C, activated protein C, protein S and protein Z, or a mixture thereof.

In a particularly advantageous fashion, the protein of the invention is the FVII, or the activated FVII (FVIIa).

In this respect, the FVII or the FVIIa can be produced according to the teaching of the document EP 0 527 063, and the summary of the method thereof is given hereinbefore. A DNA fragment, the sequence of which is that of the human FVII, is then placed under the control of the WAP promoter. For example, such a DNA sequence is listed under the sequence number 1b described in the document EP 0 200 421.

Advantageously, the FVII of the invention is activated. The FVIIa results, in vivo, from the cleavage of the zymogen with different proteases (FIXa, FXa, FVIIa) into two chains linked by a disulfide bridge. The FVIIa alone has a very poor enzymatic activity, but complexed with its cofactor, the tissue factor (TF), it triggers the coagulation process by activating the FX and the FIX.

The FVIIa exhibits a coagulant activity by 25 to 100 times higher than that of the FVII upon their interaction with the tissular factor (TF).

In an embodiment of the invention, the FVII can be activated in vitro by the Factors Xa, VIIa, IIa, IXa and XIIa.

The FVII of the invention can also be activated during the purification process thereof.

The Applicant has surprisingly noticed that the protein of interest, even if placed under the control of a promoter of a protein naturally produced in the lactoserum, such as the WAP promoter, for example, is nevertheless liable to be associated with calcium ions, and thus with the casein micelles.

Thus, the process of the invention can be used for separating recombinant proteins produced under the control of a promoter of a protein of the lactoserum.

Moreover, the process of the invention is particularly adapted to the separation of recombinant proteins produced under the control of a casein promoter.

The protein can also be selected from the group consisting of Factor VIII, alpha-1-anti-trypsin, anti-thrombin III, albumin, fibrinogen, insulin, myelin basic protein, proinsulin, plasminogen tissue activator, and antibodies, or a mixture thereof.

The process of the invention can also be used for preparing a recombinant lactic protein. In this case, it can be a matter of a lactic protein synthetized in the mammary gland of an animal of different species (Simons and al, (1987), Aug. 6-12; 328(6130):530-532). To this end transgenic lactoferrin, lactoglobulin, lysozym and/or lactalbumin can be mentioned by way of examples.

A further object of the invention is related to a non-lipidic aqueous phase of the milk comprising at least one protein liable to be obtained by the process of the invention. Advantageously, the aqueous phase is hypersaline, basic, and contains the soluble caseins and at least one further protein of interest. Hypersaline, refers preferentially to a concentration of at least 7 g/l of sodium ions or at least 18 g/l of sodium chloride, or at least 0.3 molar of sodium chloride. Preferentially, this concentration is of about 8 g/l sodium ions or of about 20 g/l of sodium chloride. Basic refers to a pH comprised between 8 to 9, and preferentially, higher than 7.8. The soluble caseins represent at least 25% of total caseins, and more preferentially, at least 50% of total caseins.

Such a phase comprises at least 50%, or advantageously from at least 60% to 80%, of the total proteins of interest to be purified comparing to the milk which was not subjected to the process steps. In a particularly advantageous ways, the non-lipidic aqueous phase comprises at least 90% of the total proteins of interest present in the milk prior to extraction.

According to a preferred embodiment of the invention, the protein of interest, present in the non-lipidic aqueous phase, is the Factor VII (FVII) or the activated Factor VII (FVIIa).

The non-lipidic aqueous phase of the invention, even if it does not contain casein micelles and insoluble calcium compounds anymore, comprises still, however, mostly impurities. Consequently, it is necessary, depending upon the individual case, to proceed to a purification of the protein in the aqueous phase.

The process of the invention can include, moreover, subsequent purification steps of the non-lipidic aqueous phase comprising the protein or proteins of interest, obtained after the step c) or, possibly, after the filtration and concentration/dialysis steps carried out after this step c).

Thus, the step c) is followed by a step d) of affinity chromatography, using a standard chromatographic system, advantageously carried out on a chromatographic column with a hydroxyapatite gel (Ca₁₀(PO₄) 6 (OH)₂) or a fluoroapatite gel (Ca₁₀(PO₄)₆F₂) support. Thus, the protein of the non-lipidic aqueous phase is retained on the support, the major part of the non-retained lactic proteins being removed. The detection is performed through absorbance measurement at λ=280 nm.

The chromatographic column is preferably equilibrated with an aqueous buffer A based on 0.025 M-0.035 M sodium phosphate, pH 7.5-8.5. The non-lipidic aqueous phase is injected onto the column, what allows the retention of the protein of interest. The non-retained fraction is removed by percolation of the buffer A, until return to baseline (RBL), what assures a good elimination of undesirable compounds, such as lactic proteins.

The elution of the protein is performed with a buffer based on a phosphate salt, such as sodium or potassium phosphate, or a mixture thereof, in a predetermined concentration, preferably representing a buffer B based on 0.25 M-0.35 M sodium phosphate, pH 7.5-8.5. The eluted fraction is collected until return to baseline.

Owing to this step, more than 90% of the total lactic proteins are eliminated, and more than 90% of proteins of interest are recovered. The purity of this eluted fraction is of about 5% in this step.

The purity is defined as being the mass ratio between the protein of interest and the total proteins present in the considered sample, fraction or eluate.

Advantageously, the specific activity of the protein or proteins is increased by a factor 10 à 25 as a result of the affinity of the protein of interest for the chromatographic support.

The eluate obtained as a result of the step d) is subsequently advantageously subjected to a tangential filtration. The tangential filtration membrane is selected by those skilled in the art depending upon the characteristics of the protein of interest. Generally speaking, a porosity limit with a pore size two times greater than the molecular weight of the protein of interest allows to filter advantageously the product. For example, a membrane with a pore size of 100 kDa allows to filter the FVII with good yields.

The aim of this step of filtration is to reduce the load especially of proteins with a molecular weight higher than that of the protein of interest and, in particular, to remove the atypical forms of the protein of interest (for example proteins in polymerised form), and the proteases liable to degrade it within a certain delay, as well.

In a very preferential way, the obtained filtered eluate is further concentrated and dialyzed. A suitable system has already been described for the step of concentration/dialysis by ultrafiltration.

According to a preferred embodiment of the invention, the process comprises at least one ion exchange chromatography step in order to purify the protein of interest, and, in particular, two successive chromatographic steps on ion exchangers. This preferably allows the removal of the remaining lactic proteins.

The choice of the ion exchanger and of the equilibrating, washing and elution buffers depends upon the nature of the protein to be purified.

This step or steps can be carried out directly after the step c), or, optionally, after the affinity chromatography and/or tangential filtration steps.

Preferably, the at least one step and the two chromatography steps are anion exchange chromatographies. More preferably, said anion exchange chromatographies are performed using weak base type chromatographic supports. The detection of the compounds is ensured through absorbance measurement at λ=280 nm.

The second chromatographic step is intended to limit a possible proteolytic degradation of the protein.

By way of example, use is made of, in the first chromatographic step of the Factor VII purification from the non-lipidic aqueous phase, a Q-Sepharose® FF gel type chromatographic support onto which is retained the Factor VII. Use is made of an aqueous elution buffer based on, preferably 0.05 M, Tris, and of, preferably 0.020 M-0.05 M, calcium chloride, pH 7.0-8.0, in order to obtain an eluate of Factor VII having an intermediary purity, that is a purity from 25% to 75%.

The eluate of FVII can further be subjected to dialysis step, as previously described, the buffer of which is a 0.15 M sodium chloride solution.

In the second chromatographic step, use is made of, for example for purifying the eluate of Factor VII obtained in the previous step, optionally diluted in order to allow it to be adsorbed again, a Q-Sepharose® FF gel type chromatographic support onto which is retained the Factor VII. Use is made of an aqueous elution buffer based on, preferably 0.05 M, Tris, and, preferably 0.005 M, calcium chloride, pH 7.0-8.0, for eluting a high-purity fraction of Factor VII, that is a purity higher than 90%.

According to a preferred embodiment of the invention, the process comprises, after the two anion exchange chromatography steps, a third anion exchange chromatography step. This step allows to formulate the protein-enriched composition, in a way to make it adapted to medical use. More preferably, said third chromatographic step is carried out using weak base type chromatographic support. The detection of the compounds is also ensured through measurement at λ=280 nm.

By way of example, the eluate obtained by the second anion exchange chromatographic step is injected, after dilution, onto a column filled up with a Q-Sepharose® FF gel type support onto which is retained the Factor VII. The Factor VII retained on the support is eluted with an aqueous buffer consisting of preferably 0.02 M Tris, and of 0.20-0.30 M sodium chloride, pH 6.5-7.5.

Consequently, the three chromatographic steps on an anion exchanger gel allow to purify further the protein of interest. In addition, they allow the concentration and formulation of the composition of the protein of interest.

According to a preferred embodiment of the invention, and when the protein of interest to be purified is a clotting factor, at least one of the three chromatographic steps on anion exchangers supports allow to activate the whole or a part of the clotting factor. Advantageously, the first chromatography allows the activation of the clotting factor.

Once the last eluate is recovered, said eluate could be submitted to a filtration step on 0.22 μm filters, to a distribution step in containers and then freezed to −30° C. and stored at this temperature.

The process of the invention can also comprise at least one of the following steps: formulation, virus inactivation and sterilization. Generally speaking, the process can comprise, prior to the affinity chromatography step, an anti-viral treatment step, which is advantageously performed with solvent/detergent, in particular in the presence of a mixture of Tween® 80 (1% w/v) and of TnBP (tri-n-butylphosphate) (0.3% v/v,), what allows to inactivate the enveloped viruses. Moreover, the eluate resulting from the second chromatographic step on anion exchangers is preferably subjected to a step of nanofiltration in order to eliminate efficiently the viruses, in particular the nonenveloped viruses, such as the parvovirus B19. It is possible to use the filters ASAHI PLANOVA™15 retaining the viruses with a size greater than 15 nm.

Further aspects and advantages of the invention will be described in the following examples, which are to be considered as illustrating and not limiting the scope of the invention.

### EXAMPLES

The hereinafter examples illustrate the application of the process of extraction and of purification of the invention for preparing a concentrate of activated Factor VII (FVIIa) from the milks of transgenic female rabbits (FVII-tg transgenic FVII).

These raw milks result from the first lactation of five females F1 (2^{nd} generation of the founder lineages). The females ware selected on the basis of the rate of lactic secretion of the FVII antigene (FVII:Ag). The STAGO (ASSERACHROM VII) kit allowed to follow the content of the human FVII from D04 to D25 (D: milking day) from the first lactation. This secretion was comparatively stable for these females (between 188 and 844 IU/ml of FVII), depending upon the female and the day of collection).

The selected purification process allowed to purify, for example, 12 mg of FVII-tg from a pool of 500 ml of raw milk. The global yield of purification is 22%.

This concentrate is pure according to the analysis by SDS-PAGE electrophoresis under non reduced condition, i.e that disulfide bridges are maintained, and exhibits a complete cleavage of the heavy and light chains under reduced conditions, what results in the complete transformation into activated FVII (FVIIa) during the process.

### Example 1

### Extraction of FVII from the Milk

500 ml of raw whole milk are diluted by 9 volumes of 0.25 M sodium phosphate buffer, pH 8.2. After stirring for 30 minutes at room temperature, the aqueous FVII-enriched phase is centrifuged at 10 OOOg for 1 hour at 15° C. (centrifuge Sorvall Evolution RC—6700 rpm—rotor SLC-6000). 6 pots of about 835 ml are necessary.

Three phases are present after centrifugation: a lipidic phase on the surface (cream), a clear non-lipidic aqueous phase enriched in FVII (phase in majority) and a white, solid, phase in pellet (precipitates of insoluble caseins and of calcium compounds).

The non-lipidic aqueous phase comprising FVII is collected by means of a peristaltic pump up to the creamy phase. The creamy phase is collected separately. The solid phase (precipitate) is removed.

The non-lipidic aqueous phase, however comprising still very low amounts of lipids, is filtered on a sequence of filters (Pall SLK7002U010ZP—prefilter of glass fibers with a pore size of 1 μm—then Pall SLK7002NXP—Nylon 66 with a pore size of 0.45 μm). At the end of filtration, the lipidic phase is passed on this sequence of filtration which retains completely the fat globules of the milk, and the filtrate is clear.

The filtered non-lipidic aqueous phase is further dialyzed on an ultrafiltration membrane (Millipore Biomax 50 kDa-0.1 m²) in order to make it compatible with the chromatography stage. The FVII with a high molecular weight of about 50 kDa does not filter through the membrane, in contrast to salts, sugars and peptides of the milk. In a first time, the solution (about 5 000 ml) is concentrated to 500 ml, then a dialysis by ultrafiltration maintaining the constant volume allows to eliminate the electrolytes and to condition the biological material for the step of chromatography. The dialysis buffer is a 0.025M sodium phosphate buffer, pH 8.2.

This non-lipidic aqueous phase comprising the FVII can be assimilated to the FVII-tg-enriched lactoserum. This preparation is stored at −30° C. prior to continuation of the process.

The global yield of recovery of the FVII by this step is very satisfactory: 90% (91% extraction with phosphate+99% dialysis/concentration).

The non-lipidic aqueous phase comprising the FVII resulting from this step is perfectly clear and is compatible with the further chromatographic steps.

Of about 93 000 IU of FVII-tg are extracted at this stage. The purity of this preparation in FVII is of the order of 0.2%.

### Example 2

### Process for Purification of FVIIa

1. Hydroxyapatite Gel Chromatography

An Amicon 90 (diameter 9 cm—cross section 64 cm²) column is filled up with BioRad Ceramic Hydroxyapatite type I gel (CHT-I). The detection is performed through absorbance measurement at λ=280 nm.

The gel is equilibrated with an aqueous buffer A consisting of a mixture of 0.025 M sodium phosphate and 0.04 M sodium chloride, pH 8.0. The whole preparation, preserved at −30° C., is thawed in a water bath at 37° C. until complete dissolution of the ice bloc, then is injected onto the gel (linear flow rate 100 cm/h, that is 105 ml/min). The non-retained fraction is eliminated by passing of a buffer consisting of 0.025 M sodium phosphate and 0.04 M sodium chloride, pH 8.2, until return to base line (RBL).

The elution of the fraction containing the FVII-tg is performed with the buffer B consisting of 0.25 M sodium phosphate and 0.4 M sodium chloride, pH 8.0. The eluted fraction is collected until return to base line.

This chromatography allows to recover more than 90% of the FVII-tg, while eliminating more than 95% of the lactic proteins. The specific activity (S.A.) is multiplied by 25. Of about 85 000 IU of FVII-tg with a purity of 4% are available at this stage.

2. 100 kDa Tangential Filtration and 50 kDa Concentration/Dialysis

The whole eluate of the preceding step is filtered in a tangential mode on a 100 kDa ultrafiltration membrane (Pall OMEGA SC 100K-0.1 m²). The FVII is filtered through a 100 kDa membrane, while the proteins with a molecular weight higher than 100 kDa can not be filtered.

Further, the filtered fraction is concentrated to about 500 ml, then dialyzed on a 50 kDa ultrafilter, already described in the Example 1. The dialysis buffer is 0.15 M sodium chloride.

At this stage of the process, the product is stored at −30° C. prior to the passage in ion exchange chromatography.

This step allowed to reduce the load of proteins with a molecular weight higher than 100 kDa and in particular of pro-enzymes. The 100 kDa membrane treatment allows to retain of about 50% of the proteins, among which the high molecular weight proteins, while 95% of the FVII-tg are filtered, that is 82 000 IU of FVII-tg.

This treatment allows to reduce the risks of proteolytic hydrolysis in the downstream steps.

3. Chromatographies on Q-Sepharose® FF Gel

These three successive chromatographies on ion exchanger gel Q-Sepharose® Fast Flow (QSFF) are performed in order to purify the active principle, to allow the activation of the FVII to activated FVII (FVIIa) and finally to concentrate and formulate the composition of FVII. The detection of the compounds is performed through absorbance measurement at λ=280 nm.

3.1 Q-Sepharose® FF 1 Step—Elution <<High Calcium>>

A column of 2.6 cm diameter (cross section 5.3 cm²) is filled up with 100 ml of Q-Sepharose® FF gel (GE Healthcare).

The gel is equilibrated with a 0.05 M Tris buffer, pH 7.5.

The whole fraction preserved at −30° C. is thawed in water bath at 37° C. until complete dissolution of the ice bloc. The fraction is diluted by ½ [v/v] with the equilibrating buffer prior to injection onto the gel (flow rate 13 ml/min, that is a linear flow rate of 150 cm/h), then the non-retained fraction is eliminated by passage of the buffer until RLB.

A first proteic fraction with a low content of FVII is eluted at 9 ml/min (that is 100 cm/h) with a buffer of 0.05 M Tris and 0.15 M sodium chloride, pH 7.5, and is subsequently eliminated.

A second FVII-rich proteic fraction is eluted at 9 ml/min (that is 100 cm/h) with a buffer of 0.05 M Tris, 0.05 M sodium chloride and 0.05 M calcium chloride, pH 7.5. The detection is performed at λ=280 nm.

This second fraction is dialyzed on a 50 kDa ultrafilter already described in the Example 1. The dialysis buffer is 0.15 M sodium chloride. This fraction is preserved at +4° C. during the night prior to 2^{nd} passage of anion exchange chromatography.

This step allows to recover 73% of the FVII (that is to say 60000 IU of FVII-tg), while eliminating 80% of associated proteins. It allows also to activate the FVII in FVIIa.

3.2 Q-Sepharose® FF 2 Step—Elution <<Low Calcium>>

A 2.5 cm diameter (4.9 cm² cross section) column is filled up with 30 ml of Q-Sepharose® FF gel (GE Healthcare).

The gel is equilibrated with buffer 0.05 M Tris, pH 7.5.

The preceding eluted fraction (second fraction), stored at +4° C., is diluted prior to injection onto the gel (flow rate 9 ml/min, that is a linear flow rate 100 cm/h).

After the injection, the gel is washed with the equilibrating buffer for the removal of the non-retained fraction.

A fraction containing very high purity FVII is eluted at 4.5 ml/min (that is 50 cm/h) in a buffer of 0.05 M Tris, 0.05 M sodium chloride and 0.005 M calcium chloride, pH 7.5.

Of about 23 000 IU of FVII-tg were purified, that is 12 mg of FVII-tg.

This step allows to remove more than 95% of the associated proteins (proteins of the milk of female rabbit).

This eluate, with a purity higher than 90%, exhibits structural and functional features close to those of the natural human FVII molecules. It is concentrated and formulated by a third passage in anion exchange chromatography.

3.3 Q-Sepharose® FF 3 Step—Elution <<Sodium>>

A 2.5 cm diameter (4.9 cm² cross section) column is filled up with 10 ml of Q-Sepharose® FF gel (GE Healthcare).

The gel is equilibrated with 0.05 M Tris buffer, pH 7.5.

The eluted, purified fraction of the preceding step is diluted by five times with purified water for injection (PWI) prior to injection on the gel (flow rate 4.5 ml/min, that is a linear flow rate of 50 cm/h).

After the injection, the gel is washed with the equilibrating buffer for the removal of the non-retained fraction.

Afterwards, the FVII-tg is eluted with a flow rate of 3 ml/min (that is 36 cm/h) with buffer of 0.02 M Tris and 0.28 M sodium chloride, pH 7.0.

A concentrate of FVII-tg was prepared with a purity higher than 95%. The product is compatible with an intravenous injection. The process has a cumulated yield of 22%, allowing to purify at least 20 mg of FVII per liter of milk used.

The Table A resumes the steps of the process according to a preferred embodiment of the invention, and gives different yields, the purity and the specific activities obtained in each step.

TABLE A

| | | Protein | | | | | |
|---|---|---|---|---|---|---|---|
| | Volume | Content | FVII:Ag | Yield FVII/ | Yield FVII/ | SA | FVII purity |
| Batch n^{o}479030 | (ml) | (mg) | (IU) content | step (%) | cumulated % | (IU/mg) | (%) |
| Pool of raw | 500 | 42750 | 103450 | 100% | 100% | 2.4 | 0.12% |
| milk | | | | | | | |
| Phosphate | 4785 | ND | 93650 | 91% | 91% | — | — |
| extraction | | | | | | | |
| Concentration/ | 667 | 29610 | 93233 | 99% | 90% | 3.1 | 0.20% |
| Dialysis | | | | | | | |
| (50 kDa UF) | | | | | | | |
| Hydroxyapatite | 2644 | 1071 | 85692 | 92% | 79% | 80.0 | 4.0% |
| eluate (CHT-I) | | | | | | | |
| Tangential | 459 | 518 | 81684 | 95% | 72% | 157.6 | 7.9% |
| Filtration | | | | | | | |
| (100 kDa UF) | | | | | | | |
| Eluate QSFF1 | 402 | 105 | 59757 | 73% | 58% | 572 | 28.6% |
| (high Ca++) | | | | | | | |
| Eluate QSFF2 | 157 | 12.8 | 22447 | 38% | 22% | 1749 | 87% |
| (low Ca++) | | | | | | | |
| Eluate QSFF3 | 42.5 | 12.7 | 21929 | 98% | 21% | 1727 | 86% |
| (Sodium) | | | | | | | |
| Final product | 50 | 12.4 | 23197 | 106% | 22% | 1878 | 94% |
| (sterilisation | | | | | | | |
| 0.2 μm | | | | | | | |

1. Process for extracting at least one protein present in milk, said protein exhibiting an affinity for the complexed or non-complexed calcium ions of said milk, comprising the following steps consisting of:
a) releasing the protein by precipitation of calcium compounds obtained by contacting the milk with a soluble salt, the anion of which is selected for its capability to form in such a medium said insoluble calcium compounds, in order to obtain in this way a protein-enriched liquid phase,
b) separating the protein-enriched liquid phase from the precipitate of calcium compounds, said liquid phase being, moreover, separated in a lipidic phase and in a non-lipidic aqueous phase comprising the protein, and
c) recovering the non-lipidic aqueous phase comprising the protein.

2. Process according to claim 1, wherein the soluble salt is a phosphate salt.

3. Process according to claim 2, wherein the phosphate salt is selected from the group consisting of sodium phosphate, lithium phosphate, potassium phosphate, rubidium phosphate and cesium phosphate, and is, in particular, sodium phosphate.

4. Process according to claim 1, wherein the soluble salt is an alkali metal oxalate, in particular sodium or potassium oxalate, or an alkali metal carbonate, in particular sodium or potassium carbonate, or a mixture thereof.

5. Process according to claim 1, wherein the soluble salt concentration in aqueous solution is comprised between 100 mM and 3 M, more preferably, between 200 mM and 500 mM and, in particular, between 200 mM and 300 mM.

6. Process according to claim 1, wherein the step b) is carried out by centrifugation.

7. Process according to claim 1, comprising, after the step c), a step of filtration of the non-lipidic aqueous phase carried out successively on filters with a decreasing porosity, preferably of 1 μm, then of 0.45 μm, followed by a step of concentration/dialysis by ultrafiltration.

8. Process according to claim 1, wherein the lipidic phase is filtered through filters with a decreasing porosity, preferably of 1 μm, then of 0.45 μm.

9. Process according to claim 1, wherein the protein is a protein not naturally present in the milk.

10. Process according to claim 1, wherein the milk is a milk of a transgenic-non-human mammal.

11. Process according to claim 10, wherein said mammal is selected among female rabbit, sheep, goat, cow, pig and mouse.

12. Process according to claim 9, wherein the protein is a clotting protein.

13. Process according to claim 12, wherein the protein is selected from the group consisting of Factor II, Factor VII, Factor IX and Factor X, and their activated forms, as well, protein C, activated protein C, protein S and protein Z, or a mixture thereof.

14. Process according to claim 9, wherein the protein is a protein comprising GLA-domains, EGF domains (epidermal growth factor) or further domains known to have a capability to fix the calcium ions.

15. Process according to claim 9, wherein the protein is a vitamin K dependent protein.

16. Process according to claim 9, wherein the protein is a calcium dependent protein.

17. Process according to claim 9, wherein the protein is selected from the group consisting of Factor VIII, alpha-1-anti-trypsin, anti-thrombin III, albumin, fibrinogen, insulin, myelin basic protein, proinsuline, tissue plasminogen activator, and antibodies, or a mixture thereof.

18. Process according to claim 9, wherein the protein is transgenic lactoferrin, lactoglobulin, lysozyme and/or lactalbumin.

19. Non-lipidic aqueous phase of milk characterized in that it is hypersaline, basic and that it contains soluble caseins and at least one further protein, liable to be obtained by the process according to claim 1.

20. Process according to claim 1, wherein the step c) is followed by a step d) of affinity chromatography, the elution of the protein being performed with a buffer based on a phosphate salt at a predetermined concentration.

21. Process according to claim 20, wherein the affinity chromatography is carried out on a chromatographic column the support of which is hydroxyapatite gel (Ca₁₀(PO₄)₆(OH)₂) or a fluoroapatite gel (Ca₁₀(PO₄)₆F₂).

22. Process according to claim 20, wherein the eluate obtained from the step d) is further subjected to a tangential filtration.

23. Process according to claim 1, comprising at least one step of ion exchange chromatography, and, in particular, two successive chromatography steps on ion exchangers carried out directly after the step c).

24. Process according to claim 23, wherein the at least one step and the two steps of chromatography are anion exchange chromatographies.

25. Process according to claim 24, comprising, after the two anion exchange chromatography steps, a third anion exchange chromatography step.

26. Process according to claim 20, comprising, prior to the affinity chromatography step, an anti-viral treatment step carried out by solvent/detergent.

27. Process according to claim 23, wherein the eluate resulting from the second chromatography step on anion exchanger is subjected to a nanofiltration step.
